(19) United States
(12) Patent Application Publication
(10) Pub. No.: US 2019/0021744 A1
(43) Pub. Date: Jan. 24, 2019

(54) Hand-Wound Tourniquet

(71) Applicants: Jeffery S, Will, Phoenix, AZ (US); Hans W. Burkardt, Gilbert, AZ (US); Thomas Vernon Stanley, Chandler, AZ (US); Sapna Patel, Scottsdale, AZ (US); Clifford Loader, Scottsdale, AZ (US); Lawrence Slater, Gilbert, AZ (US)

(72) Inventors: Jeffery S. Will, Phoenix, AZ (US); Hans W. Burkardt, Gilbert, AZ (US); Thomas Vernon Stanley, Chandler, AZ (US); Sapna Patel, Scottsdale, AZ (US); Clifford Loader, Scottsdale, AZ (US); Nicholas D Wallick, Gilbert, AZ (US); Lawrence Slater, Gilbert, AZ (US)

(21) Appl. No.: 16/069,275

(22) PCT Filed: Jan. 12, 2017
(86) PCT No.: PCT/IB2017/050171

§ 371 Date: Jul. 11, 2018

Related U.S. Application Data
(60) Provisional application No. 62/277,766, filed on Jan. 12, 2016.

(51) Int. Cl. A61B 17/132

(52) U.S. Cl. CPC A61B 17/1327

(57) ABSTRACT

A hand-wound tourniquet is an apparatus that stops blood flow of an injured appendage as quickly as possible and with minimal effort. The apparatus applies constant pressure against the limb without any manual input. The apparatus includes a housing, a strap, a hook, a reeling shaft, a handle, and a cam-locking mechanism. The reeling shaft traverses into the housing and is rotatably and slidably mounted to the housing. The handle is positioned external to the housing and is torsionally connected to the reeling shaft. The strap traverses into the housing. The cam-locking mechanism is operatively integrated into the torsional connection between the reeling shaft and the handle, wherein the longitudinal motion of the reeling shaft is used to actuate the cam-locking mechanism in order to prevent the rotation of the reeling shaft. The apparatus preferably includes a mount that accommodates the curvature of a limb.

The current application is a 371 of international Patent Cooperation Treaty (PCT) application PCT/IB2017/050171 filed on Jan. 12, 2017.

The current application is a continuation-in-part (CIP) application of international Patent Cooperation Treaty (PCT) application PCT/IB2017/050171 filed on Jan. 12, 2017.

The PCT application PCT/IB2017/05017 claims a priority to a U.S. provisional application Ser. No. 62/277,766 filed on Jan. 12, 2016.

### FIELD OF THE INVENTION

The present invention generally relates to the medical apparatus. More specifically, the present invention is a hand-wound tourniquet that quickly deploys strap and requires minimal effort to both tighten the strap and maintain the pressure of the strap.

### BACKGROUND OF THE INVENTION

Use of tourniquets have been controversial over the years due to the correlation between the user of tourniquets and amputation and nerve damage. In recent years, however, the popularity of tourniquets has increased. Over the past fourteen years, the wars in Afghanistan and Iraq have been particularly demanding on the limbs of young soldiers. The survival rates of these young soldiers have been greatly increased as a result of better control of the loss of blood and the faster arrival of the injured soldiers to a surgical center within the hour.

The tourniquets used to save lives today utilize a concept that dates back centuries. The tourniquets of today utilizes a piece of material with a stick. The stick is then inserted under the material and turned to tighten. The military and first responders use this concept but with a nylon strap and a plastic twist rod to tighten the strap. Even with this improvement, which applies more pressure on the injured limb, tourniquets still require skill to properly mount and force to tighten.

Therefore, an objective of the present invention is to tighten a nylon strap while lessening the amount of time and effort to wrap around an injured limb. The present invention is manually tightened to the proper tension in order to shut the flow of blood and may be locked so that the necessary pressure is applied to the injured limb without any additional input.

### BRIEF DESCRIPTION OF THE DRAWINGS

FIG. 1 is a front perspective view of the preferred embodiment of the present invention, wherein the handle is in an upright orientation and the cam-locking mechanism is unlocked.

FIG. 2 is a front perspective view of the preferred embodiment of the present invention, wherein the handle is in a prone orientation and the cam-locking mechanism is locked.

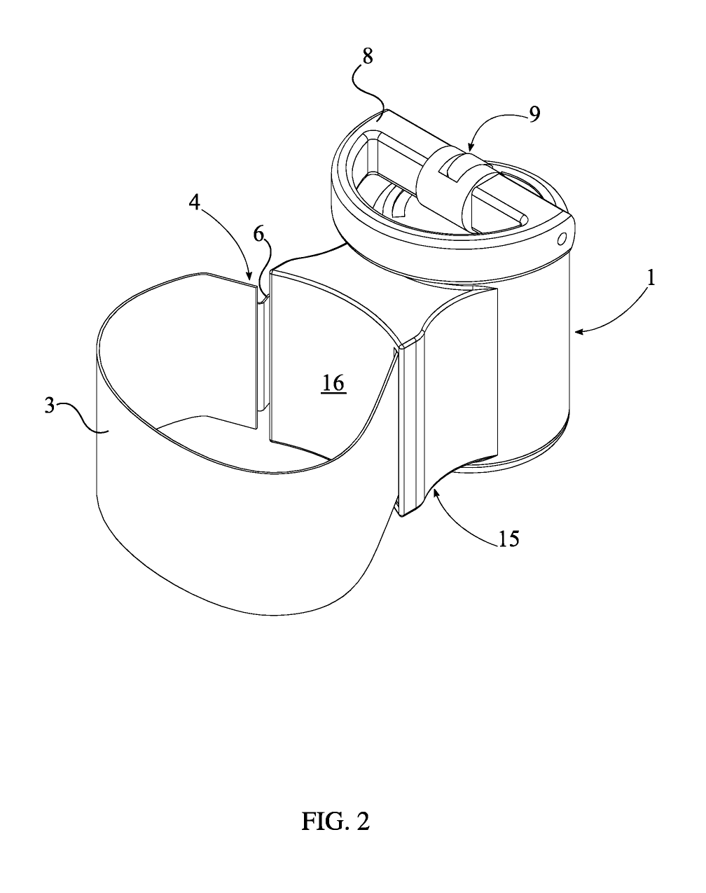

FIG. 3 is a rear perspective view of the preferred embodiment of the present invention, wherein the handle is in an upright orientation and the cam-locking mechanism is unlocked.

FIG. 4 is a front perspective view of the present invention without the housing.

FIG. 5 is a front perspective exploded view exploded view of the preferred embodiment of the present invention without the strap and the hook.

FIG. 6 is a bottom exploded view of the preferred embodiment of the present invention without the strap and the hook

FIG. 7 is a front perspective view of an embodiment of the present invention, wherein the padding is fixed to the mount.

FIG. 8 is a front perspective view of an embodiment of the present invention without the strap and the hook, wherein the slot traverses through the housing.

### DETAILED DESCRIPTION OF THE INVENTION

All illustrations of the drawings are for the purpose of describing selected versions of the present invention and are not intended to limit the scope of the present invention.

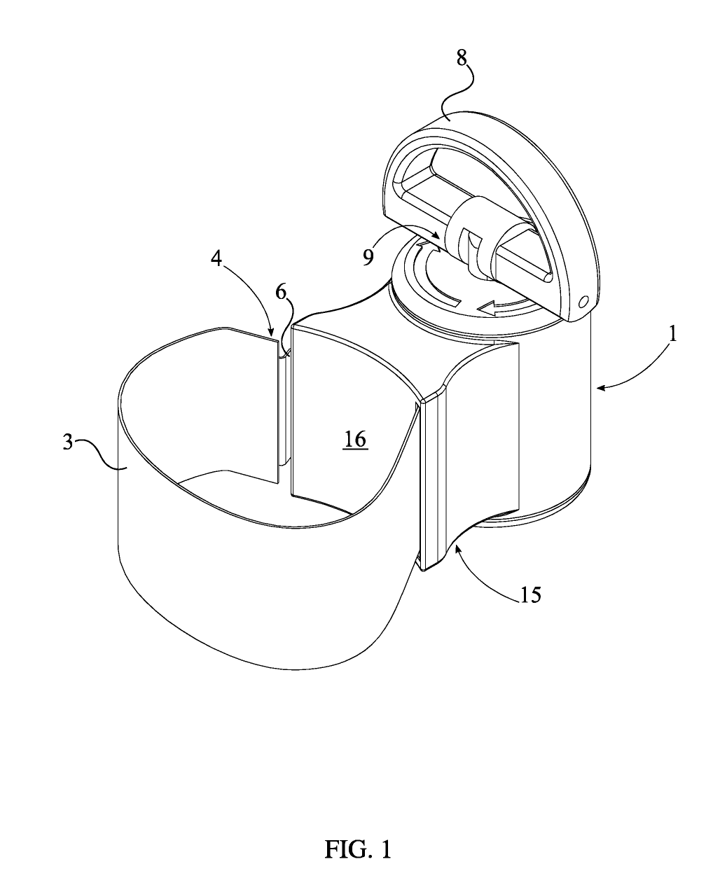

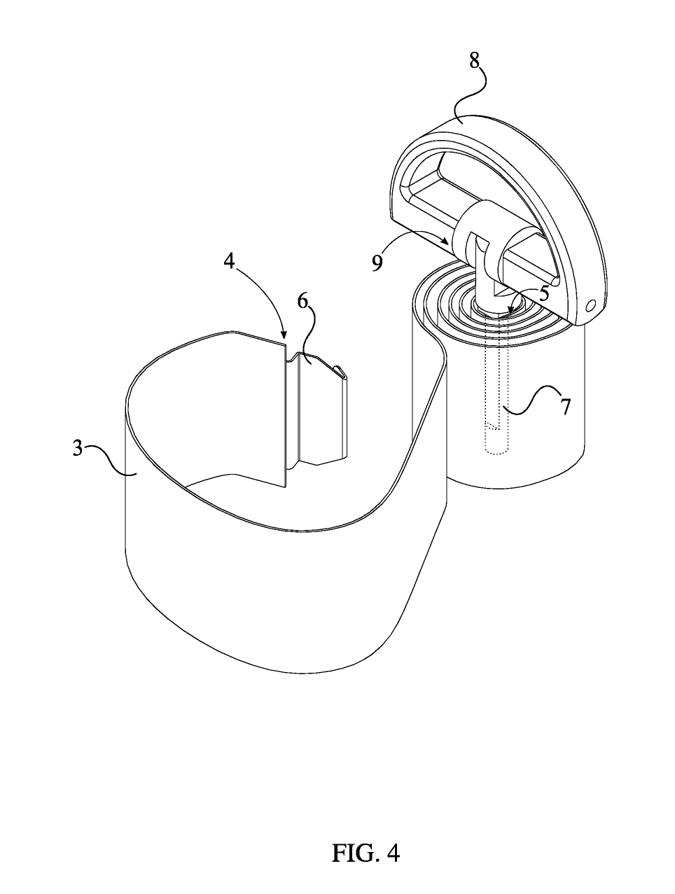

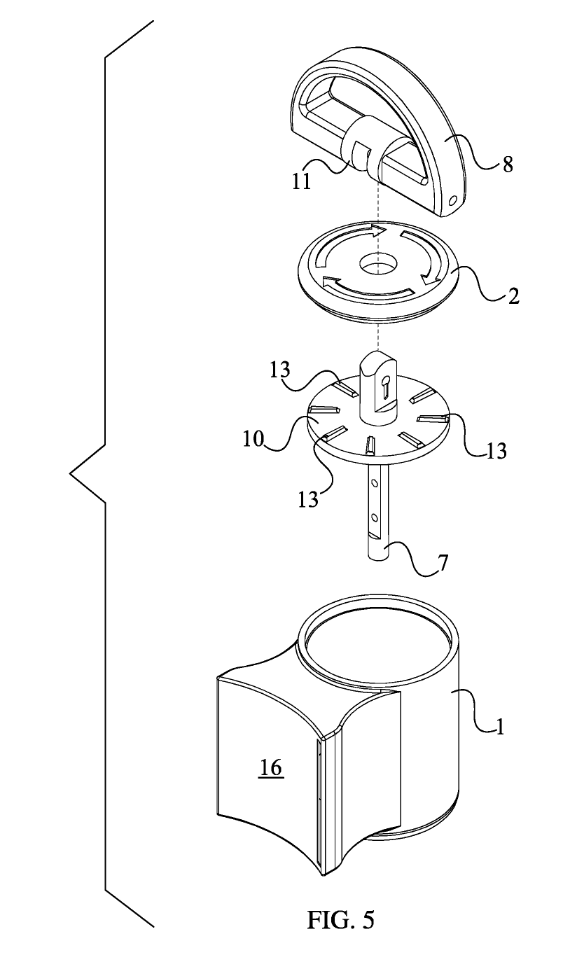

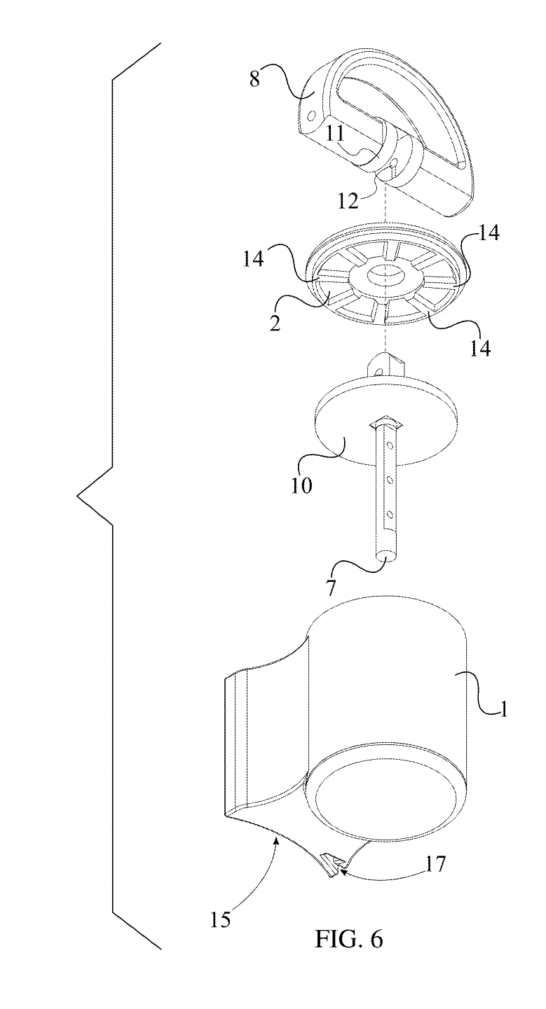

The present invention is a medical apparatus that quickly stops the blood flow of an appendage with minimal effort by a user. The present invention applies the necessary amount of pressure to an engaged, injured limb for a continuous period of time without a user having to continuously apply force upon the present invention. The present invention better mounts onto an injured limb, facilitating the wrapping of the apparatus around the limb. As illustrated in FIG. 1, FIG. 5, and FIG. 6, the present invention comprises a housing 1, a strap 3, a hook 6, a reeling shaft 7, a handle 8, and a cam-locking mechanism 9. The housing 1 stores the strap 3, upholds the reeling shaft 7 and handle 8, and positions the cam-locking mechanism 9. The strap 3 wraps around a limb, adjacent the injury along the limb, and applies pressure upon the limb. The strap 3 comprises a first strap end 4 and a second strap end 5. The first strap end 4 allows the hook 6 to move externally about the housing 1, and the second strap end 5, as shown in FIG. 4, attaches the strap 3 to the reeling shaft 7. The hook 6 connects the second strap end 5 to the housing 1 so that the strap 3 loops around an injured limb. The reeling shaft 7 extends and retracts the strap 3 upon the engagement of the handle 8. The cam-locking mechanism 9 prevents the strap 3 from extending and retracting about the reeling shaft 7, thereby applying constant pressure against the injured limb as needed.

The overall configuration of the aforementioned components allows a user to attach the present invention onto a user's injured limb and stop further blood loss from the injury. The reeling shaft 7 traverses into the housing 1 so that the strap 3 is stored within the housing 1, as shown in the exploded views of FIG. 5 and FIG. 6. Furthermore, the reeling shaft 7 is rotatably and slidably mounted to the housing 1. The rotation of the reeling shaft 7 allows the strap 3 to wind and unwind into the housing 1. The sliding of the reeling shaft 7 corresponds to the locking and unlocking of the cam-locking mechanism 9. The locking and unlocking of the cam-locking mechanism 9 allows for and prevents the rotation of the reeling shaft 7 within the housing 1, respectively. The handle 8 is positioned external to the housing 1 and is torsionally connected to the reeling shaft 7 so that the user may grasp onto the handle 8 and maneuver the reeling shaft 7 via the handle 8. This arrangement allows the user to extend the length of the strap 3 outside of the housing 1 and retract the strap 3 into the housing 1. More specifically, as the strap 3 traverses into the housing 1, the first strap end 4 is positioned external to the housing 1, and the second strap end 5 is positioned within the housing 1. The hook 6 is fixed onto the first strap end 4, and the second strap end 5 is fixed to the reeling shaft 7. The arrangement allows the hook 6 to clip onto the housing 1 in order to create a loop with the strap 3, while the strap 3 remains connected to the reeling shaft 7. The user engages the cam-locking mechanism 9 via the handle 8 in order to secure the length of the strap 3 outside of the housing 1. More specifically, the cam-locking mechanism 9 is operatively integrated into the torsional connection between the reeling shaft 7 and the handle 8, wherein longitudinal motion of the reeling shaft 7 is used to actuate the cam-locking mechanism 9 in order to prevent the rotation of the reeling shaft 7.

In order to prevent the length of the strap 3 from changing, the cam-locking mechanism 9 comprises a stopping plate 10 and a roller 11, illustrated in FIG. 5 and FIG. 6. The stopping plate 10 locks and unlocks the rotation of the reeling shaft 7 within the housing 1. The roller 11 connects the cam-locking mechanism 9 and the handle 8. In order for the roller 11 to engage with the handle 8, the roller 11 is positioned external to the housing 1 and tangentially contacts the housing 1, which allows the roller 11 to roll against the housing 1. Furthermore, an off-center axle 12 of the roller 11 is rotatably connected to the reeling shaft 7, and the handle 8 is torsionally connected to the roller 11. This configuration allows the handle 8 to rotate the roller 11 and consequently allows the off-center axle 12 to convert the rotational motion of the roller 11 into linear motion with the reeling shaft 7. In an alternate embodiment of the present invention, a pin is positioned along the off-center axle 12 and allows for the rotatable connection between the handle 8 and the roller 11. The stopping plate 10 is positioned within the housing 1 so that the stopping plate 10 comes into contact with the housing 1. More specifically, an engageable portion 2 of the housing 1 is positioned adjacent to the stopping plate 10, allowing for the engagement of the stopping plate 10 and the housing 1. The stopping plate 10 is axially fixed along the reeling shaft 7 so that the stopping plate 10 rotates simultaneously with the reeling shaft 7. In the preferred embodiment of the present invention, a hole traverses through the center of the stopping plate 10, and a block is axially fixed along the reeling shaft 7. The hole of the stopping plate 10 is a square-like hole, which allows the block to be fitted within the hole of stopping plate 10. An external tooth lock washer is laterally fixed along the reeling shaft 7 so that the external tooth lock washer prevents the stopping plate 10 from slipping past the block. The external tooth lock washer is positioned adjacent the block and within the hole of the stopping plate 10 such that the inner rim of the external tooth lock washer surrounds the reeling shaft 7 and the teeth of the external tooth lock washer presses against the stopping plate 10. Upon the engagement of the stopping plate 10 and the engageable portion 2, both the reeling shaft 7 and the stopping plate 10 stop rotating.

In the preferred embodiment of the present invention, the cam-locking mechanism 9 further comprises a first set of locking teeth 13 and a second set of locking teeth 14. The first set of locking teeth 13, as shown in FIG. 5, lock and unlock the rotation of the reeling shaft 7 and, consequently, the handle 8. The first set of locking teeth 13 is connected onto the stopping plate 10 and is radially positioned around the reeling shaft 7. This arrangement accommodates the torsional movement of the stopping plate 10 about the reeling shaft 7. The second set of locking teeth 14 engages with the first set of locking teeth 13 and secures orientation of the stopping plate 10 about the reeling shaft 7. The second set of locking teeth 14 is positioned within the housing 1 and connected onto the engageable portion 2 of the housing 1, as shown in FIG. 6. Moreover, the second set of locking teeth 14 is radially positioned around the reeling shaft 7 so that the second set of locking teeth 14 accommodates the torsional movement of the stopping plate 10 and the first set of locking teeth 13. The stopping plate 10 presses against the engageable portion 2 of the housing 1, wherein the stopping plate 10 and the engageable portion 2 of the housing 1 are arranged in a locked configuration. In the locked configuration, the length of the strap 3 is fixed. In order to extend or retract the strap 3, the user raises the handle 8. The user twists the handle 8 in an upright orientation with respect to the housing 1 until the user achieves the desired length and pressure of the strap 3 about an engaged, injured limb. The user flips the handle 8 about the roller 11 so that the handle 8 is in a prone orientation with respect to the housing 1, and the first set of locking teeth 13 and the second set of locking teeth 14 engage each other and consequently are in a locked configuration with each other.

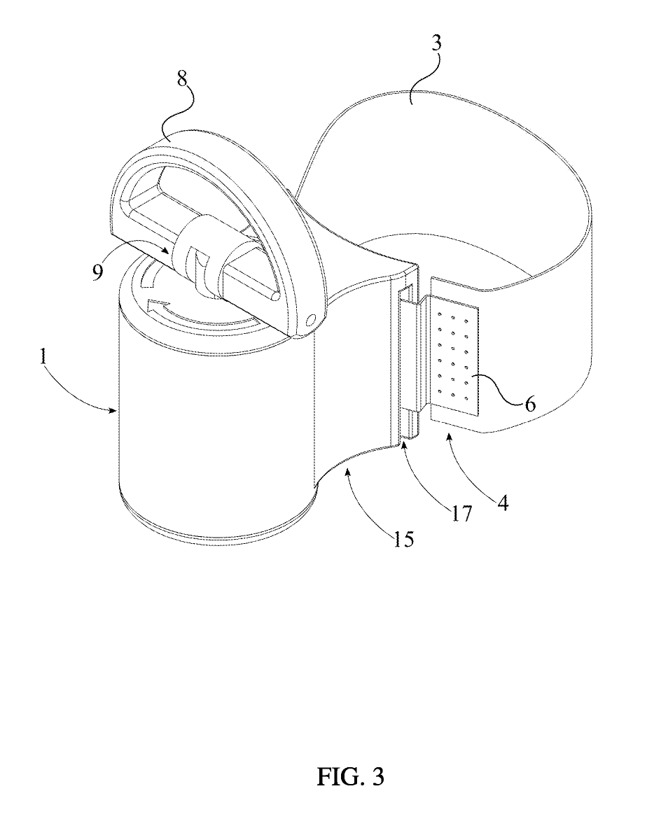

In order to fasten the present invention onto an injured limb in a quicker fashion, a mount 15 surrounds and directly presses upon the injured limb of the user. The mount 15 further comprises an appendage-bracing portion 16 and hook-clasping portion 17, as shown in FIG. 1 and FIG. 3. The appendage-bracing portion 16 surrounds a portion of an injured limb, and the hook-clasping portion 17 secures the hook 6 to the mount 15. The user preferably engages the hook 6 with the hook-clasping portion 17 before the tightening or loosening of the strap 3 about the injured limb. In order to directly press the mount 15 upon the injured limb, the housing 1 is connected adjacent to the mount 15.

More specifically, the appendage-bracing portion 16 is positioned opposite to the housing 1. The strap 3 traverses out of the mount 15, adjacent to the appendage-bracing portion 16 so that the position of the injured limb against the appendage-bracing portion 16 is maintained while the strap 3 is tightened and loosened. In order for the first strap end 4 to loop around the entirety of the injured limb that presses against the appendage-bracing portion 16, the hook-clasping portion 17 is positioned adjacent to the appendage-bracing portion 16, opposite to the strap 3. In the preferred embodiment of the present invention, the hook-clasping portion 17 is a hole, wherein a cross-section of the hole is a barb shape. The hole traverses into the mount 15, parallel to the reeling shaft 7, so that the hook 6 engages to the barb shape. In an alternate embodiment, the hole traverses through the mount 15 so that the hook 6 may slide into the hole from either side. This arrangement prevents the strap 3 from twisting and consequently applying inconsistent pressure about the injured limb. In an alternate embodiment of the present invention, the hook-clasping portion 17 is a hole, wherein a cross-section of the hole is a circular shape, and the hook 6 is engaged to the circular shape. In various embodiments of the present invention, the cross-section of the hole may be a variety of shapes that accommodate the hook 6, however the parallel arrangement between the hole and the reeling shaft 7 needs to be preserved by the present invention.

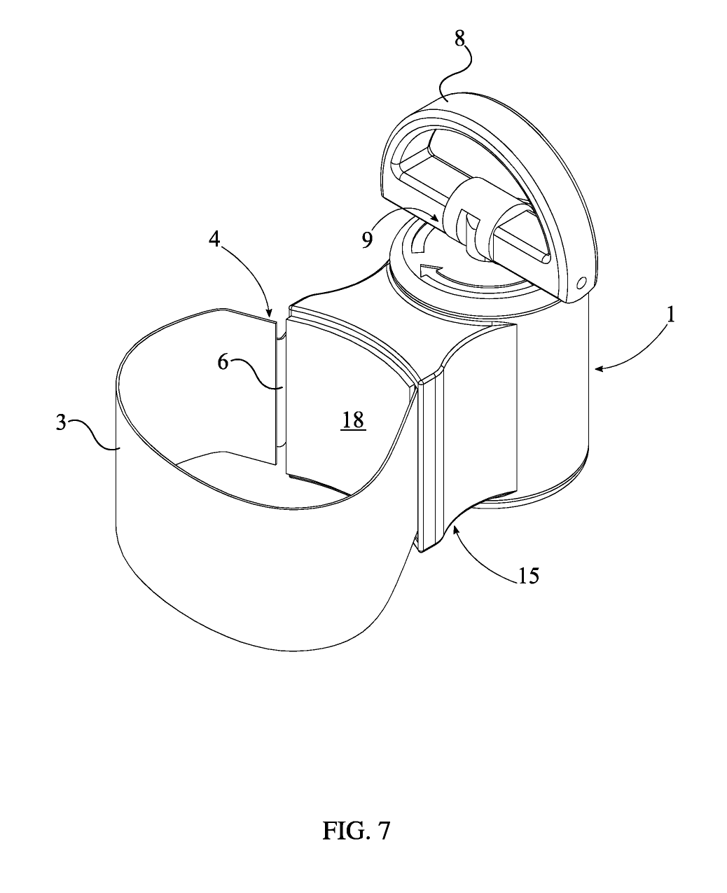

In the preferred embodiment of the present invention, the mount 15 further comprises a padding 18. The padding 18 lessens the impact of the injured limb against the appendage-bracing portion 16, so that the limb does not bruise or get injured any further, while still maintaining the pressure applied by the strap 3. The padding 18 is attached to the appendage-bracing portion 16, positioned opposite the housing 1, and traverses across the appendage-bracing portion 16. This arrangement, shown in FIG. 7, cushions the user's injured limb against the entirety of the appendage-bracing portion 16.

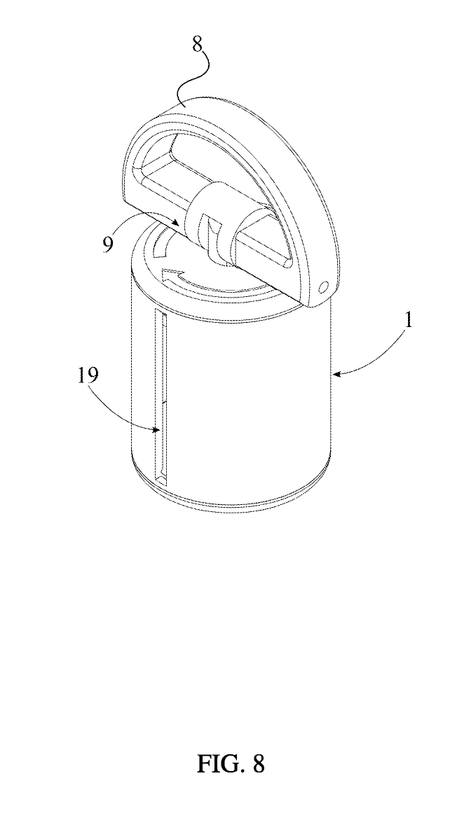

In an alternate embodiment of the present invention, a slot 19 accommodates the both the strap 3 and hook 6. As shown in FIG. 8, the slot 19 traverses into the housing 1, similar to that of the hook-clasping portion 17 of the mount 15. However, in this alternate embodiment, the strap 3 is positioned through the slot 19 and the hook 6 engages to the slot 19. More specifically, the strap 3 presses against a lateral edge of the slot 19 as the strap 3 exits the slot 19, and the hook 6 latches onto the opposite lateral edge of the slot 19 as the strap 3 loops around an injured limb. In this embodiment, the housing 1 is not connected adjacent to the mount 15 as the slot 19 is the point of exit so that the strap 3 may fully encompass the injured limb and more pressure may be applied.

Although the invention has been explained in relation to its preferred embodiment, it is to be understood that many other possible modifications and variations can be made without departing from the spirit and scope of the invention as hereinafter claimed.

What is claimed is:

1. A hand-wound tourniquet comprises:
a housing;
a strap;
a hook;
a reeling shaft;
a handle;
a cam-locking mechanism;
the strap comprises a first strap end and a second strap end;
the reeling shaft traversing into the housing;
the reeling shaft being rotatably and slidably mounted to the housing;
the handle being positioned external to the housing;
the handle being torsionally connected to the reeling shaft;
the strap traversing into the housing;
the first strap end being positioned external to the housing;
the hook being fixed onto the first strap end;
the second strap end being positioned within the housing;
the second strap end being fixed to the reeling shaft; and
the cam-locking mechanism being operatively integrated into the torsional connection between the reeling shaft and the handle, wherein longitudinal motion of the reeling shaft is used to actuate the cam-locking mechanism in order to prevent rotation of the reeling shaft.

2. The hand-wound tourniquet as claimed in claim 1 comprises:
the cam-locking mechanism comprises a stopping plate and a roller;
the roller being positioned external to the housing;
the roller tangentially contacting the housing;
an off-center axle of the roller being rotatably connected to the reeling shaft;
the handle being torsionally connected to the roller;
the stopping plate being positioned within the housing;
the stopping plate being axially fixed along the reeling shaft; and
an engageable portion of the housing being positioned adjacent to the stopping plate.

3. The hand-wound tourniquet as claimed in claim 2 comprises:
the cam-locking mechanism further comprises a first set of locking teeth;
the first set of locking teeth being connected onto the stopping plate; and
the first set of locking teeth being radially positioned around the reeling shaft.

4. The hand-wound tourniquet as claimed in claim 2 comprises:
the cam-locking mechanism further comprises a second set of locking teeth;
the second set of locking teeth being positioned within the housing;
the second set of locking teeth being connected onto the engageable portion of the housing; and
the second set of locking teeth being radially positioned around of the reeling shaft.

5. The hand-wound tourniquet as claimed in claim 2 comprises:
wherein the stopping plate and the engageable portion of the housing is arranged in a locked configuration; and
the stopping plate being pressed against the engageable portion of the housing.

6. The hand-wound tourniquet as claimed in claim 1 comprises:
a mount;
the mount comprises an appendage-bracing portion and a hook-clasping portion;
the housing being connected adjacent to the mount;
the appendage-bracing portion being positioned opposite to the housing;
the strap traversing out of the mount, adjacent to the appendage-bracing portion; and
the hook-clasping portion being positioned adjacent to the appendage-bracing portion, opposite to the strap.

7. The hand-wound tourniquet as claimed in claim 6 comprises:
the hook-clasping portion being a hole, wherein a cross-section of the hole is a barb shape;
the hole traversing into the mount, parallel to the reeling shaft; and
the hook being engaged to the barb shape.

8. The hand-wound tourniquet as claimed in claim 6 comprises:
the hook-clasping portion being a hole, wherein a cross-section of the hole is a circular shape;
the hole traversing into the mount, parallel to the reeling shaft; and
the hook being engaged to the circular shape.

9. The hand-wound tourniquet as claimed in claim 6 comprises:
the mount further comprises a padding;
the padding being attached onto the appendage-bracing portion; and
the padding being positioned across the appendage-bracing portion.

10. The hand-wound tourniquet as claimed in claim 1 comprises:
a slot;
the slot traversing into the housing; and
the strap being positioned through the slot.

11. The hand-wound tourniquet as claimed in claim 8 comprises:
the hook being engaged to the slot.

12. A hand-wound tourniquet comprises:
a housing;
a strap;
a hook;
a reeling shaft;
a handle;
a cam-locking mechanism;
the strap comprises a first strap end and a second strap end;
the cam-locking mechanism comprises a stopping plate and a roller;
the reeling shaft traversing into the housing;
the reeling shaft being rotatably and slidably mounted to the housing;
the handle being positioned external to the housing;
the handle being torsionally connected to the reeling shaft;
the strap traversing into the housing;
the first strap end being positioned external to the housing;
the hook being fixed onto the first strap end;
the second strap end being positioned within the housing;
the second strap end being fixed to the reeling shaft;
the cam-locking mechanism being operatively integrated into the torsional connection between the reeling shaft and the handle, wherein longitudinal motion of the reeling shaft is used to actuate the cam-locking mechanism in order to prevent rotation of the reeling shaft;
the roller being positioned external to the housing;
the roller tangentially contacting the housing;
an off-center axle of the roller being rotatably connected to the reeling shaft;
the handle being torsionally connected to the roller;
the stopping plate being positioned within the housing;
the stopping plate being axially fixed along the reeling shaft; and
an engageable portion of the housing being positioned adjacent to the stopping plate.

13. The hand-wound tourniquet as claimed in claim 12 comprises:
the cam-locking mechanism further comprises a first set of locking teeth;
the first set of locking teeth being connected onto the stopping plate; and
the first set of locking teeth being radially positioned around the reeling shaft.

14. The hand-wound tourniquet as claimed in claim 12 comprises:
the cam-locking mechanism further comprises a second set of locking teeth;
the second set of locking teeth being positioned within the housing;
the second set of locking teeth being connected onto the engageable portion of the housing; and
the second set of locking teeth being radially positioned around of the reeling shaft.

15. The hand-wound tourniquet as claimed in claim 12 comprises:
wherein the stopping plate and the engageable portion of the housing is arranged in a locked configuration; and
the stopping plate being pressed against the engageable portion of the housing.

16. The hand-wound tourniquet as claimed in claim 12 comprises:
a mount;
the mount comprises an appendage-bracing portion and a hook-clasping portion;
the housing being connected adjacent to the mount;
the appendage-bracing portion being positioned opposite to the housing;
the strap traversing out of the mount, adjacent to the appendage-bracing portion; and
the hook-clasping portion being positioned adjacent to the appendage-bracing portion, opposite to the strap.

17. The hand-wound tourniquet as claimed in claim 16 comprises:
the hook-clasping portion being a hole, wherein a cross-section of the hole is a barb shape;
the hole traversing into the mount, parallel to the reeling shaft; and
the hook being engaged to the barb shape.

18. The hand-wound tourniquet as claimed in claim 16 comprises:
the hook-clasping portion being a hole, wherein a cross-section of the hole is a circular shape;
the hole traversing into the mount, parallel to the reeling shaft; and
the hook being engaged to the circular shape.

19. The hand-wound tourniquet as claimed in claim 16 comprises:
the mount further comprises a padding;
the padding being attached onto the appendage-bracing portion; and
the padding being positioned across the appendage-bracing portion;
the hook being engaged to the slot

20. The hand-wound tourniquet as claimed in claim 12 comprises:
a slot;
the slot traversing into the housing; and
the strap being positioned through the slot.
